(19) United States
(12) Patent Application Publication
(10) Pub. No.: US 2025/0040980 A1
(43) Pub. Date: Feb. 6, 2025

(54) PLASMA GUIDE WIRE

(71) Applicant: ASAHI INTECC CO., LTD., Seto-shi (JP)

(72) Inventors: Naozumi IWATA, Seto-shi (JP); Tomoki ICHIKAWA, Seto-shi (JP); Shumpei YOSHITAKE, Seto-shi (JP)

(73) Assignee: ASAHI INTECC CO., LTD., Seto-shi (JP)

(21) Appl. No.: 18/923,199

(22) Filed: Oct. 22, 2024

Related U.S. Application Data
(63) Continuation of application No. PCT/JP2023/009235, filed on Mar. 10, 2023.

(30) Foreign Application Priority Data
May 17, 2022 (JP) 2022-080672

(51) Int. Cl. A61B 18/04; A61B 18/00

(52) U.S. Cl. CPC A61B 18/042; A61B 2018/00351; A61B 2018/00577; A61B 2018/00595

(57) ABSTRACT

A plasma guide wire includes a conductive core shaft configured to be connected to a high-frequency generator, a conductive coil body that surrounds a part of the core shaft on a distal end side of the core shaft, and a distal tip that includes a conductive material and fixes a distal end of the core shaft and a distal end of the coil body, the distal tip being configured to receive a high frequency wave from the high-frequency generator via the core shaft. An outer surface of the distal tip includes (i) a proximal end side region located on a side of the coil body and (ii) a distal end side region located distally relative to the proximal end side region. The distal end side region has an electric resistance value that is lower than an electric resistance value of the proximal end side region.

This application is a bypass continuation of International Application No. PCT/JP2023/009235 filed Mar. 10, 2023, which is based upon and claims the benefit of priority from Japanese Patent Application No. 2022-080672 filed May 17, 2022, the entire contents of the prior applications being incorporated herein by reference.

### TECHNICAL FIELD

The disclosed embodiments relate to a plasma guide wire.

### BACKGROUND ART

As methods for treating arrhythmia resulting in abnormal heartbeat rhythm or chronic total occlusion (CTO) resulting from occlusion of a blood vessel by a lesion, plasma ablation therapies have been recently known, in which a living body tissue is ablated (cauterized) using a plasma flow. For example, Patent Literature 1 discloses a device usable for such plasma ablation therapies. The device described in Patent Literature 1 includes an energy supply apparatus equipped with an energy delivery electrode having a first surface area, a sheath equipped with an energy return electrode having a second surface area larger than the first surface area, and an energy generator that outputs electric power to each of these electrodes.

### CITATION LIST

### Patent Literature

Patent Literature 1: US 2019/0223948 A

### SUMMARY

### Technical Problems

In the device described in Patent Literature 1, when a high voltage is applied to the energy delivery electrode and the energy return electrode, streamer corona discharge is generated around the energy delivery electrode, and this streamer corona discharge can ablate a living body tissue in the vicinity of the energy delivery electrode. However, in the device described in Patent Literature 1, since the discharge phenomenon occurs uniformly over the entire energy delivery electrode, the electric field intensity around the energy delivery electrode becomes uniform. Thus, the device described in Patent Literature 1 has had a problem of a possibility that not only the target site of the living body tissue (e.g., CTO) but also the entire living body tissue surrounding the energy delivery electrode is ablated, i.e., a possibility that a site other than the target site is ablated. Such a problem is not limited to guide wires for vascular systems but is common to all plasma guide wires that are inserted into a living body lumen such as the lymphatic system, the biliary system, the urinary system, the respiratory system, the digestive system, the secretory gland, and the reproductive organ for the plasma ablation therapy. Furthermore, plasma guide wires have required improved operability and low manufacturing cost.

The disclosed embodiments were made to solve at least a part of the above problems, and an object of the disclosed embodiments is to provide a plasma guide wire that allows ablation to be localized.

### Solution to Problems

The disclosed embodiments were made to solve at least a part of the above problems and can be embodied as the following aspects, among other aspects.

(1) According to one aspect of the disclosed embodiments, a plasma guide wire is provided. This plasma guide wire includes a conductive core shaft, a conductive coil body that surrounds a part of the core shaft on a distal end side of the core shaft, and a distal tip made of conductive material, which fixes a distal end of the core shaft and a distal end of the coil body and receives a high frequency wave from a high-frequency generator electrically connected to the core shaft. An outer surface of the distal tip includes (i) a proximal end side region located on a side of the coil body and (ii) a distal end side region located distally relative to the proximal end side region. The distal end side region has an electric resistance value that is lower than an electric resistance value of the proximal end side region.

According to this configuration, the outer surface of the distal tip includes the proximal end side region located on the side of the coil body, and the distal end side region located on the distal end side relative to the proximal end side region, and the distal end side region has a lower electric resistance value than that of the proximal end side region. Thus, when a high frequency wave is applied from the high-frequency generator to the distal tip that functionally serves as a distal end electrode, plasma can be generated concentratively on the distal end side region of the distal tip. In other words, the intensity of the electric field generated in association with the streamer corona discharge can be enhanced on the distal end side region compared to the proximal end side region in the distal tip. As a result, a portion other than the target site (e.g., CTO) in the living body tissue, e.g., a living body tissue located in the vicinity of the proximal end side region of the distal tip can be prevented from being ablated. According to this configuration, an insulating member that insulates the plasma guide wire on the proximal end side relative to the distal tip can be prevented from being damaged compared to the conventional configuration in which the electric field intensity around the distal tip is uniform during discharge. As a result, the durability of the plasma guide wire can be improved. In this way, this configuration makes it possible to provide a plasma guide wire that is excellent in durability and allows ablation to be localized.

(2) In the plasma guide wire with the above configuration, a portion of the outer surface of the distal tip on the distal end side region may have a corner portion that is sharper than a remaining portion of the outer surface of the distal tip in the distal end side region. According to this configuration, since a part of the outer surface on the distal end side region of the distal tip has a corner portion sharper than the other portion, plasma can be concentratively generated particularly on the corner portion of the distal end side region of the distal tip when a high frequency wave is applied to the distal tip from a high-frequency generator. In other words, the intensity of the electric field generated in association with the streamer corona discharge can be enhanced particularly on the corner portion of the distal end side region of the distal tip. As a result, this configuration makes it possible to provide a plasma guide wire that allows ablation to be further localized.

(3) In the plasma guide wire with the above configuration, the proximal end side region need not have any corner portion. According to this configuration, since the proximal end side region of the distal tip does not have the corner portion, plasma can be generated more concentratively on the corner portion of the distal end side region compared to a configuration with a corner portion disposed on the proximal end side region. As a result, this configuration makes it possible to provide a plasma guide wire that allows ablation to be further localized.

The disclosed embodiments can be embodied in various aspects, such as a plasma guide wire, a plasma ablation system equipped with a plasma guide wire and a radio frequency (RF) generator, a guide wire that ablates (cauterizes) a living body tissue using heat instead of plasma, and a method for producing a plasma guide wire or a guide wire.

### BRIEF DESCRIPTION OF DRAWINGS

FIG. 1 is an explanatory view illustrating a sectional configuration of a plasma guide wire.

FIG. 2 is an enlarged sectional view illustrating a distal end side of the plasma guide wire.

FIG. 3 is an enlarged perspective view illustrating the distal end side of the plasma guide wire.

FIGS. 4A and 4B are explanatory views illustrating an electric field intensity distribution during discharge.

FIGS. 5A and 5B are explanatory diagrams illustrating a plasma guide wire according to the second embodiment.

FIG. 6 is an enlarged sectional view illustrating a distal end side of a plasma guide wire according to the third embodiment.

FIG. 7 is an enlarged sectional view illustrating a distal end side of a plasma guide wire according to the fourth embodiment.

FIG. 8 is an enlarged sectional view illustrating a distal end side of a plasma guide wire according to the fifth embodiment.

FIG. 9 is an enlarged sectional view illustrating a distal end side of a plasma guide wire according to the sixth embodiment.

FIG. 10 is an enlarged sectional view illustrating a distal end side of a plasma guide wire according to the seventh embodiment.

FIG. 11 is an enlarged sectional view illustrating a distal end side of a plasma guide wire according to the eighth embodiment.

FIG. 12 is an explanatory view illustrating a sectional configuration of a plasma guide wire according to the ninth embodiment.

### DETAILED DESCRIPTION OF EMBODIMENTS

### First Embodiment

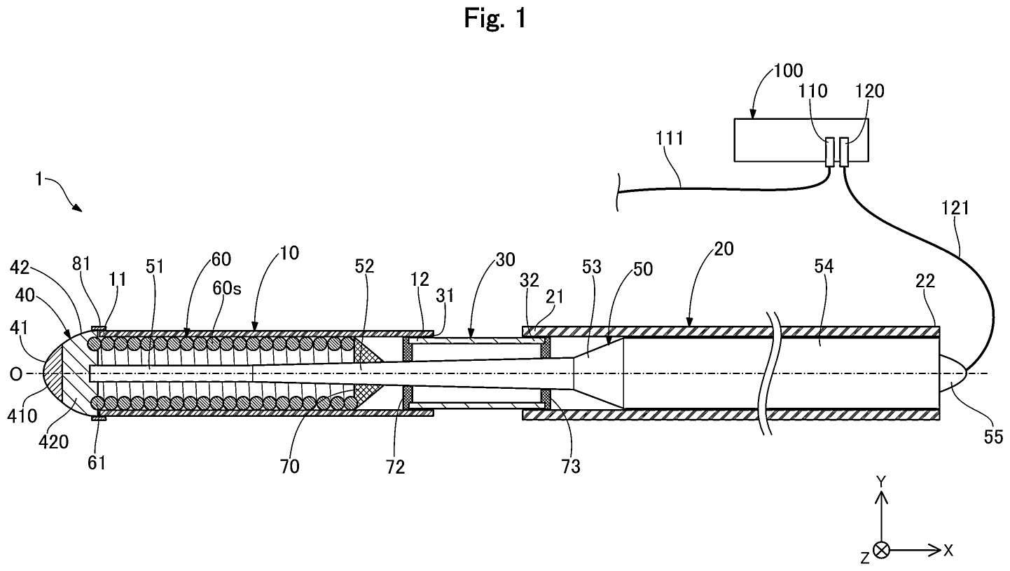

FIG. 1 is an explanatory view illustrating a sectional configuration of a plasma guide wire 1. The plasma guide wire 1 is a device that is used for the purpose of recanalizing chronic total occlusion (CTO) or treating mild to moderate stenosis, significant stenosis, arrhythmia, or the like, by ablating (cauterizing) a living body tissue using a plasma flow. Hereinafter, the disclosed embodiments will be explained with reference to a case where the plasma guide wire 1 is used for recanalizing CTO in a blood vessel, but the plasma guide wire 1 may be inserted not only into the vascular system but also into living body lumens such as the lymphatic system, the biliary system, the urinary system, the respiratory system, the digestive system, the secretory gland, and the reproductive organ.

In FIG. 1, the axis passing through the center of the plasma guide wire 1 is represented by an axis line O (dashed-dotted line). In the example of FIG. 1, the axis line O coincides with the axis passing through the center of each constituent member of the plasma guide wire 1, i.e., a first tube 10, a second tube 20, a third tube 30, a distal tip 40, a core shaft 50, and a coil body 60. However, the axis line O may be inconsistent with the center axis of each constituent member of the plasma guide wire 1. In FIG. 1, XYZ axes orthogonal to each other are illustrated. The X axis corresponds to a longitudinal direction of the plasma guide wire 1, the Y axis corresponds to a height direction of the plasma guide wire 1, and the Z axis corresponds to a width direction of the plasma guide wire 1. The left side (−X axis direction) of FIG. 1 is referred to as a “distal end side” of the plasma guide wire 1 and each constituent member, and the right side (+X axis direction) of FIG. 1 is referred to as a “proximal end side” of the plasma guide wire 1 and each constituent member. Of both ends in the longitudinal direction (X axis direction), one end located on the distal end side is referred to as a “distal end”, and the other end located on the proximal end side is referred to as a “proximal end”. The distal end and its vicinity are referred to as a “distal end portion”, and the proximal end and its vicinity are referred to as a “proximal end portion”. The distal end side is inserted into a living body, and the proximal end side is operated by an operator such as a surgeon. These definitions are common to the figures following FIG. 1.

The plasma guide wire 1 has an elongated outer shape and includes the first tube 10, the second tube 20, the third tube 30, the distal tip 40, the core shaft 50, the coil body 60, a coil fixation portion 70, a first fixation portion 72, a second fixation portion 73, and a distal end marker 81.

The distal tip 40 functionally serves as a distal end electrode. The distal tip 40 is a conductive member that causes discharge with another electrode not illustrated by a high-frequency wave applied from an RF generator 100. The other electrode is disposed in another device not illustrated. The other device may have any configuration. For example, the other device may be a catheter equipped with another electrode disposed on its distal end portion and with the plasma guide wire 1 inserted therethrough, or another guide wire equipped with another electrode disposed on its distal end portion, or a pad having another electrode.

The distal tip 40 is disposed on the frontmost end side of the plasma guide wire 1 (in other words, the distal end portion of the plasma guide wire 1). The distal tip 40 has an outer shape with a diameter reduced from the proximal end side to the distal end side for smoothening progress of the plasma guide wire 1 in a blood vessel. The distal tip 40 fixes a distal end portion 11 of the first tube 10, the distal end portion of the core shaft 50, and a distal end portion 61 of the coil body 60. The distal tip 40 will be described later in detail.

The core shaft 50 is a conductive member that constitutes the center axis of the plasma guide wire 1. The core shaft 50 has an elongated outer shape extending in the longitudinal direction of the plasma guide wire 1. The core shaft 50 includes a small diameter portion 51, a first tapered portion 52, a second tapered portion 53, and a large diameter portion 54 from the distal end toward the proximal end. The small diameter portion 51 is a portion where the outer diameter of the core shaft 50 is the smallest, and has an almost columnar shape with a substantially constant outer diameter from the distal end to the proximal end. The first tapered portion 52 is disposed between the small diameter portion 51 and the second tapered portion 53 and has an outer shape with a diameter reduced from the proximal end side to the distal end side. The second tapered portion 53 is disposed between the first tapered portion 52 and the large diameter portion 54 and has an outer shape with an outer diameter reduced from the proximal end side to the distal end side at an inclination angle different from that of the first tapered portion 52. The large diameter portion 54 is a portion where the outer diameter of the core shaft 50 is the largest and has an almost columnar shape with a substantially constant outer diameter from the distal end to the proximal end. A proximal end portion 55 of the large diameter portion 54 is a portion where a proximal end surface of the large diameter portion 54 bulges.

In the first embodiment, “substantially constant” is synonymous with “approximately constant” and means that the diameter is approximately constant while accepting fluctuations due to manufacturing error or the like. In the first embodiment, the “outer diameter” and the “inner diameter” refer to a length of the longest portion in any transverse section when the transverse section of the member (or the inner cavity) is elliptical.

The RF generator 100 is an apparatus that outputs a high frequency wave (high frequency power) between a first terminal 110 and a second terminal 120, and is also referred to as a high-frequency generator. The proximal end portion 55 of the core shaft 50 of the plasma guide wire 1 is connected with a second cable 121. The second cable 121 is a conductive electric wire. The second cable 121 extends from the second terminal 120 of the RF generator 100 to electrically connect the RF generator 100 to the plasma guide wire 1. The above-described other device having the other electrode is connected with a first cable 111. The first cable 111 is a conductive electric wire. The first cable 111 extends from the first terminal 110 of the RF generator 100 to electrically connect the RF generator 100 to the other device. The first cable 111 and the second cable 121 may have a cable connector (a connection terminal to physically and electrically connect cables).

The coil body 60 is conductive and arranged so as to surround a part of the core shaft 50 on the distal end side. In the example of FIG. 1, the coil body 60 is arranged so as to surround, in the core shaft 50, the small diameter portion 51 and a part of the first tapered portion 52 on the distal end side. The coil body 60 is formed by spirally winding a conductive wire 60s. The coil body 60 may be a single-thread coil formed by winding one wire in a single-thread form; a multi-thread coil formed by winding a plurality of wires in a multi-thread form; a single-thread strand coil formed by winding, in a single-thread form, a strand with a plurality of twisted wires; or a multi-thread strand coil formed by winding, in a multi-thread form, a plurality of strands with a plurality of twisted wires. The core shaft 50 and the coil body 60 are also collectively referred to as a “guide wire main body”.

The first tube 10 is a hollow cylindrical tubular body made of an insulating resin. The first tube 10 is disposed on the proximal end side relative to the distal tip 40 to cover the distal end side of the guide wire main body. In the example of FIG. 1, the first tube 10 covers, in the guide wire main body, the outer periphery of the coil body 60 and a part of the first tapered portion 52 of the core shaft 50 on the distal end side, exposed from the coil body 60 on the proximal end side relative to the coil body 60. The inner peripheral surface of the first tube 10 is in contact with the outer peripheral surface of the coil body 60. A thickness of the first tube 10 may be arbitrarily determined.

The second tube 20 is a hollow cylindrical tubular body made of an insulating resin. The second tube 20 is disposed on the proximal end side relative to the third tube 30 to cover the proximal end side of the guide wire main body. In the example of FIG. 1, the second tube 20 covers, in the guide wire main body, the proximal end portion of the first tapered portion 52, the second tapered portion 53, and the large diameter portion 54 in the core shaft 50. The proximal end portion 55 of the large diameter portion 54 is not covered with the second tube 20 and is exposed to the outside. The inner peripheral surface of the second tube 20 is in contact with the outer peripheral surface of the large diameter portion 54. A thickness of the second tube 20 may be arbitrarily determined.

The third tube 30 is a hollow cylindrical tubular body made of an insulating resin. The third tube 30 is disposed between the first tube 10 and the second tube 20 to cover an intermediate portion of the guide wire main body. In other words, the third tube 30 covers a part that is located in an intermediate portion of the guide wire main body and is covered with neither the first tube 10 nor the second tube 20. In the example of FIG. 1, the third tube 30 covers a part of the first tapered portion 52 of the core shaft 50 in the guide wire main body. A thickness of the third tube 30 may be arbitrarily determined.

As illustrated in FIG. 1, a distal end portion 31 of the third tube 30 is joined to a proximal end portion 12 of the first tube 10. A proximal end portion 32 of the third tube 30 is joined to a distal end portion 21 of the second tube 20. In the example of FIG. 1, an outer peripheral surface of the distal end portion 31 of the third tube 30 is joined to an inner peripheral surface of the proximal end portion 12 of the first tube 10. Similarly, an outer peripheral surface of the proximal end portion 32 of the third tube 30 is joined to an inner peripheral surface of the distal end portion 21 of the second tube 20. That means, both end portions of the third tube 30 are arranged to overlap with the proximal end portion 12 of the first tube 10 and the distal end portion 21 of the second tube 20. A portion other than the both end portions of the third tube 30 is not covered with the first tube 10 or the second tube 20 and is exposed to the outside. The first tube 10, the second tube 20, and the third tube 30 may be joined by using any bonding agent such as an epoxy adhesive. Thus, the joined body for the first, second, and third tubes 10, 20, and 30 according to the first embodiment has a shape that the intermediate portion provided with the third tube 30 is constricted.

The coil fixation portion 70 is a member that fixes the proximal end portion of the coil body 60 and a part of the first tapered portion 52 of the core shaft 50. The first fixation portion 72 is a member that is disposed on the distal end portion 31 of the third tube 30 to fix the distal end portion 31 of the third tube 30, the proximal end portion 12 of the first tube 10, and the guide wire main body (specifically, a part of the first tapered portion 52). The second fixation portion 73 is a member that is disposed on the proximal end portion 32 of the third tube 30 to fix the proximal end portion 32 of the third tube 30, the distal end portion 21 of the second tube 20, and the guide wire main body (specifically, a part of the first tapered portion 52).

The distal end marker 81 is insulative and colored in an arbitrary color, and functionally serves as a mark indicating the position of the distal tip 40. The distal end marker 81 is an annular member arranged so as to surround the outer peripheral surface of the first tube 10 on the distal end portion 11 of the first tube 10.

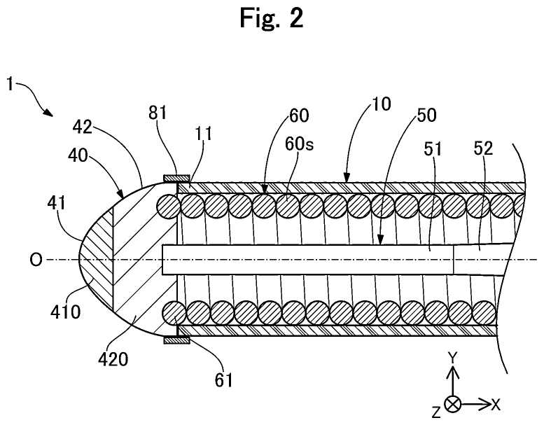

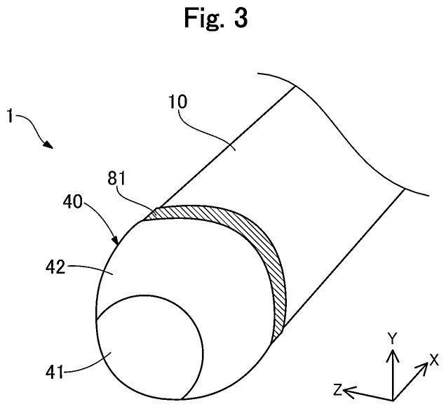

FIG. 2 is an enlarged sectional view illustrating a distal end side of the plasma guide wire 1. FIG. 3 is an enlarged perspective view illustrating a distal end side of the plasma guide wire 1. As illustrated in FIG. 2, the distal tip 40 according to the first embodiment has a first member 410 and a second member 420. The first member 410 is a hemispherical member disposed on the frontmost end side of the plasma guide wire 1. The second member 420 is a truncated conical member disposed on the proximal end side relative to the first member 410 (in other words, the side of the coil body 60 in the distal tip 40). A part of the second member 420 on the proximal end side fixes a distal end portion of the core shaft 50, the distal end portion 61 of the coil body 60, and the distal end portion 11 of the first tube 10. In the example of FIG. 2, the distal end portion of the core shaft 50 and the distal end portion 61 of the coil body 60 are embedded in a part of the second member 420 on the proximal end side. The distal end of the first tube 10 is joined to a proximal end surface of the second member 420.

The proximal end surface of the first member 410 and the distal end surface of the second member 420 have the same area, and the first member 410 and the second member 420 have a hemispherical outer shape as a whole. In the longitudinal section of the plasma guide wire 1 illustrated in FIG. 2, the interface between the first member 410 and the second member 420 is perpendicular to the center axis O. Such a distal tip 40 can be fabricated by brazing or welding the hemispherical first member 410 to the truncated conical second member 420. The distal tip 40 may be fabricated by soldering or plating the hemispherical first member 410 onto the distal end surface of the truncated conical second member 420. The second member 420 may be formed by melting the distal end portion of the core shaft 50 using laser or the like. The second member 420 is made of a conductive metallic material, such as a chromium-molybdenum steel, a nickel-chromium-molybdenum steel, a stainless steel such as SUS304, and a nickel-titanium alloy. The first member 410 is made of a conductive metal material having an electric resistance value lower than of the second member 420, such as gold (Au), silver (Ag), and platinum (Pt).

Herein, as illustrated in FIG. 2 and FIG. 3, the outer surface of the first member 410 on the outer surface of the distal tip 40 is also referred to as a distal end side region 41, and a portion not covered with the distal end marker 81 (in other words, the outer surface of the second member 420 exposed to the outside) on the outer surface of the second member 420 is also referred to as a proximal end side region 42. According to this configuration, the proximal end side region 42 is located on the side of the coil body 60, and the distal end side region 41 is located on the distal end side relative to the proximal end side region 42. In the distal tip 40 according to the first embodiment, both the distal end side region 41 and the proximal end side region 42 have a smooth surface shape without any partially-sharpened portion (corner portion). As described above, the first member 410 is made of a metal material having an electric resistance value lower than of the second member 420. Thus, the distal end side region 41 on the outer surface of the distal tip 40 has an electric resistance value lower than of the proximal end side region 42. The electric resistance value of the distal end side region 41 refers to the electric resistance value of the metal material constituting the first member 410, and the electric resistance value of the proximal end side region 42 refers to the electric resistance value of the metal material constituting the second member 420.

To increase the intensity of the electric field generated in association with streamer corona discharge described later in the distal end side region 41 compared to the proximal end side region 42, it is preferable that the surface area of the distal end side region 41 is smaller than the surface area of the proximal end side region 42 (FIG. 3). When streamer corona discharge is generated between the distal tip 40 and another electrode disposed on another device, the total surface area of the distal end side region 41 and the proximal end side region 42 (in other words, the surface area of the portion exposed to the outside, i.e., the surface area of the portion not covered with the distal end marker 81, on the outer surface of the distal tip 40) needs to be smaller than the surface area of the portion exposed to the outside on the outer surface of the other electrode in order to generate discharge on the side of the distal tip 40 (the vicinity of the distal tip 40) rather than the other electrode.

Returning to FIG. 1, the description will be continued. The first tube 10, the second tube 20, the third tube 30, and the distal end marker 81 may be made of any insulating material, e.g., a copolymer of tetrafluoroethylene and perfluoroalkoxy ethylene (PFA); a polyolefin such as polyethylene, polypropylene, and ethylene-propylene copolymer; a polyester such as polyethylene terephthalate; polyvinyl chloride; an ethylene-vinyl acetate copolymer; a crosslinked ethylene-vinyl acetate copolymer; a thermoplastic resin such as polyurethane; a polyamide elastomer; a polyolefin elastomer; a silicone rubber; a latex rubber; a super engineering plastic such as polyetheretherketone, polyetherimide, polyamide-imide, polysulfone, polyimide, and polyethersulfone. The first tube 10, the second tube 20, the third tube 30, and the distal end marker 81 may be made of a same material, or may be made of different materials depending on the performance required for the plasma guide wire 1 (e.g., flexibility, torquability, and shape maintainability of the distal end portion).

The core shaft 50 may be made of any conductive material, such as a chromium-molybdenum steel, a nickel-chromium-molybdenum steel, a stainless steel such as SUS304, and a nickel-titanium alloy. The coil fixation portion 70, the first fixation portion 72, and the second fixation portion 73 may be formed by using any bonding agent such as an epoxy adhesive.

With the plasma guide wire 1 of FIG. 1 to FIG. 3, an operator delivers the plasma guide wire 1 to the vicinity of an ablation target site (e.g., CTO) in a living body tissue, and then causes the RF generator 100 to output a high frequency wave (high frequency power) while the distal end side region 41 of the distal tip 40 is positioned in the vicinity of the target site. Then, due to a potential difference between the distal tip 40 of the plasma guide wire 1 and another electrode of another device, streamer corona discharge occurs between the distal tip 40 and the other electrode. This streamer corona discharge makes it possible to ablate the target site in the vicinity of the distal tip 40 of the plasma guide wire 1, particularly the distal end side region 41.

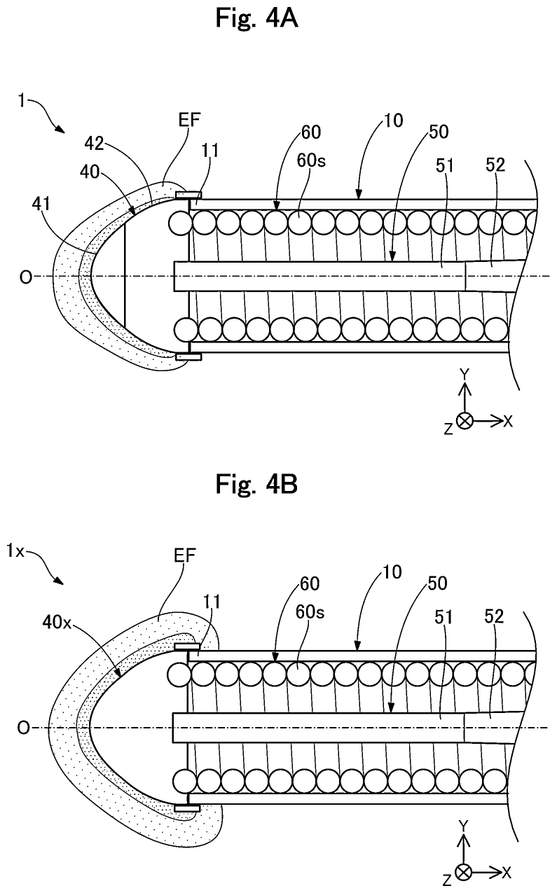

FIGS. 4A and 4B are an explanatory view illustrating an electric field intensity distribution during discharge. FIG. 4A illustrates an electric field intensity distribution EF of the plasma guide wire 1 according to the first embodiment explained with reference to FIG. 1 to FIG. 3. FIG. 4B illustrates an electric field intensity distribution EF of a plasma guide wire 1x in a comparative example. The plasma guide wire 1x in the comparative example has the same configuration as that of the plasma guide wire 1 described with reference to FIG. 1 to FIG. 3 except that the plasma guide wire 1x includes a distal tip 40x made of a single material (in other words, a distal tip 40x that does not include the distal end side region 41 and the proximal end side region 42). Similarly to the core shaft 50, the distal tip 40x is made of any conductive material (e.g., a chromium-molybdenum steel, a nickel-chromium-molybdenum steel, a stainless steel such as SUS304, and a nickel-titanium alloy). In FIG. 4A and FIG. 4B, hatching of each portion constituting the plasma guide wire 1 is omitted to clearly explain each portion.

In FIG. 4A and FIG. 4B, the portion with thick dot hatching indicates a higher electric field intensity than of the portion with thin dot hatching. As is evident from FIG. 4A, in the plasma guide wire 1 according to the first embodiment explained with reference to FIG. 1 to FIG. 3, the intensity of the electric field generated around the distal tip 40 in association with the streamer corona discharge is higher on the distal end side region 41 than on the proximal end side region 42 in the distal tip 40. In the plasma guide wire 1 according to the first embodiment, the electric field intensity distribution EF does not extend to the distal end portion 11 of the first tube 10. On the other hand, as is evident from FIG. 4B, in the plasma guide wire 1x according to the comparative example, the intensity of the electric field generated around the distal tip 40 in association with the streamer corona discharge is uniform over the entire distal tip 40x (the whole part from the distal end side to the proximal end side). In the plasma guide wire 1x according to comparative example, the electric field intensity distribution EF extends to the distal end portion 11 of the first tube 10.

As described above, in the plasma guide wire 1 according to the first embodiment, the outer surface of the distal tip 40 includes the proximal end side region 42 located on the side of the coil body 60, and the distal end side region 41 located on the distal end side relative to the proximal end side region 42, and the distal end side region 41 has a lower electric resistance value than of the proximal end side region 42. Thus, when a high frequency wave is applied from the RF generator 100 (high-frequency generator) to the distal tip 40 that functionally serves as a distal end electrode, plasma can be generated concentratively on the distal end side region 41 of the distal tip 40, as illustrated in FIG. 4A. In other words, the intensity of the electric field generated in association with the streamer corona discharge can be enhanced on the distal end side region 41 than on the proximal end side region 42 in the distal tip 40. As a result, a portion other than the target site (e.g., CTO) in the living body tissue, e.g., a living body tissue located in the vicinity of the proximal end side region 42 of the distal tip 40 can be prevented from being ablated. By the plasma guide wire 1 according to the first embodiment, the first tube 10 (insulating member) that insulates the plasma guide wire on the proximal end side relative to the distal tip 40 can be prevented from being damaged compared to the conventional configuration illustrated in FIG. 4B where the electric field intensity around the distal tip 40 is uniform during discharge. As a result, the durability of the plasma guide wire 1 can be improved. In this way, the configuration according to the first embodiment makes it possible to provide the plasma guide wire 1 that is excellent in durability and allows ablation to be localized.

### Second Embodiment

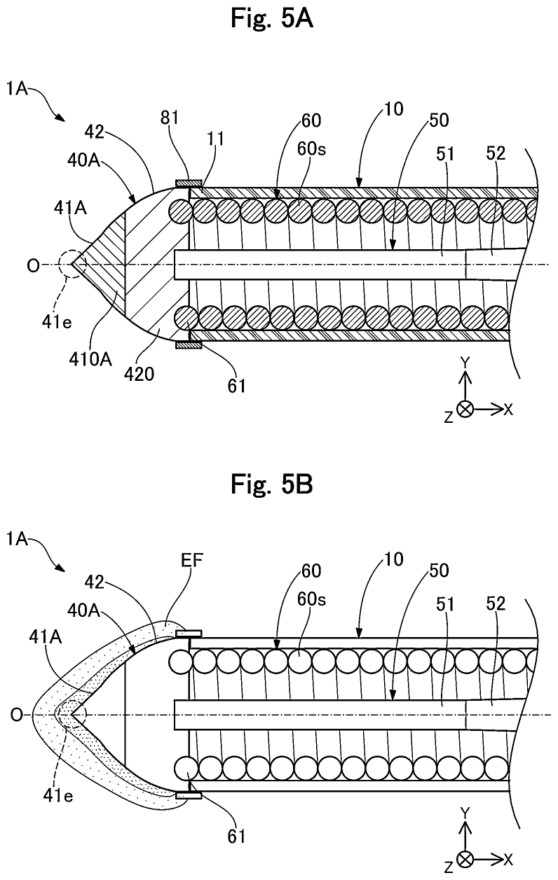

FIGS. 5A and 5B are an explanatory view illustrating a plasma guide wire 1A according to the second embodiment. FIG. 5A is an enlarged sectional view illustrating a distal end side of the plasma guide wire 1A. FIG. 5B illustrates an electric field intensity distribution EF of the plasma guide wire 1A. The plasma guide wire 1A according to the second embodiment includes a distal tip 40A instead of the distal tip 40 in the configuration according to the first embodiment. The distal tip 40A has a first member 410A instead of the first member 410.

The first member 410A is a conical member disposed on the frontmost end side of the plasma guide wire 1A. The material for the first member 410A and the method for fabricating the distal tip 40A having the first member 410A (means for brazing, welding, soldering, plating, melting, etc.) are the same as those in the first embodiment. The outer surface of the first member 410A in the outer surface of the distal tip 40A is also referred to as a distal end side region 41A. Similarly to the first embodiment, the distal end side region 41A is located on the distal end side relative to the proximal end side region 42. As illustrated in FIG. 5A, a part of the outer surface of the distal end side region 41A (portion surrounded by the circular dashed line frame) is provided with a corner portion 41e sharper than the other portion of the outer surface (portion outside the circular dashed line frame). In the example of FIG. 5A, the distal end of the corner portion 41e is oriented in the same direction as of the center axis O of the core shaft 50. As described in the first embodiment, the proximal end side region 42 has a smooth surface shape without any partially-sharpened corner portion.

As is evident from FIG. 5B, in the plasma guide wire 1A according to the second embodiment, the intensity of the electric field generated around the distal tip 40A in association with the streamer corona discharge is enhanced particularly on the corner portion 41e in the distal end side region 41. Also in the plasma guide wire 1A according to the second embodiment, the electric field intensity distribution EF does not extend to the distal end portion 11 of the first tube 10.

As described above, the configuration of the distal tip 40A can be variously modified, and the distal tip 40A may have the distal end side region 41A provided with the corner portion 41e. In the example of FIG. 5A, the corner portion 41e has a symmetrical shape in the height direction (Y axis direction) of the plasma guide wire 1A with respect to the center axis O in the longitudinal section. However, the corner portion 41e has an asymmetrical shape in the height direction (Y axis direction) of the plasma guide wire 1A with respect to the center axis O. The plasma guide wire 1A according to the second embodiment as described above can also exhibit the same effects as those in the first embodiment described above.

In the plasma guide wire 1A according to the second embodiment, since a part of the outer surface on the distal end side region 41A of the distal tip 40A has the corner portion 41e sharper than the other portion, plasma can be concentratively generated particularly on the corner portion 41e of the distal end side region 41A of the distal tip 40A when a high frequency wave is applied to the distal tip 40A from the RF generator 100, as illustrated in FIG. 5B. In other words, the intensity of the electric field generated in association with the streamer corona discharge can be enhanced particularly on the corner portion 41e in the distal end side region 41A of the distal tip 40A. As a result, the configuration according to the second embodiment makes it possible to provide the plasma guide wire 1A that allows ablation to be further localized.

In the plasma guide wire 1A according to the second embodiment, since the proximal end side region 42 of the distal tip 40A does not have the corner portion, plasma can be generated more concentratively on the corner portion 41e of the distal end side region 41A compared to the configuration with a corner portion disposed on the proximal end side region 42. As a result, the configuration according to the second embodiment makes it possible to provide the plasma guide wire 1A that allows ablation to be further localized.

### Third Embodiment

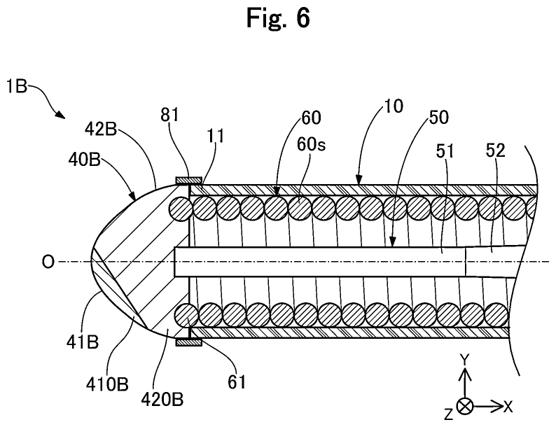

FIG. 6 is an enlarged sectional view illustrating a distal end side of a plasma guide wire 1B according to the third embodiment. The plasma guide wire 1B according to the third embodiment includes a distal tip 40B instead of the distal tip 40 in the configuration according to the first embodiment. The distal tip 40B has a first member 410B instead of the first member 410, and a second member 420B instead of the second member 420.

The first member 410B is disposed on the frontmost end side of the plasma guide wire 1B, and the second member 420B is disposed on the proximal end side relative to the first member 410B. The second member 420B has a truncated conical shape with an inclined upper surface, and the first member 410B has an irregular hemispherical shape that can fit into the upper surface of the second member 420B to form a hemisphere. As a result, in the longitudinal section of the plasma guide wire 1B illustrated in FIG. 6, the interface between the first member 410B and the second member 420B is not perpendicular to the center axis O but is oriented so as to form an acute angle smaller than 90° with the center axis O. The material for the first member 410B, the material for the second member 420B, and the method for fabricating the distal tip 40B (means for brazing, welding, soldering, plating, melting, etc.) are the same as those in the first embodiment. Similarly to the first embodiment, a distal end side region 41B is located on the distal end side relative to a proximal end side region 42B. Both the distal end side region 41B and the proximal end side region 42B have a smooth surface shape without any partially-sharpened corner portion.

As described above, the configuration of the distal tip 40B can be variously modified, and the distal end side region 41B may be disposed on any portion of the outer surface of the distal tip 40B as long as the distal end side region 41B is located on the distal end side relative to the proximal end side region 42B and the distal end side region 41B has an electric resistance value lower than of the proximal end side region 42B. The plasma guide wire 1B according to the third embodiment described above can also exhibit the same effects as those of the first embodiment described above.

### Fourth Embodiment

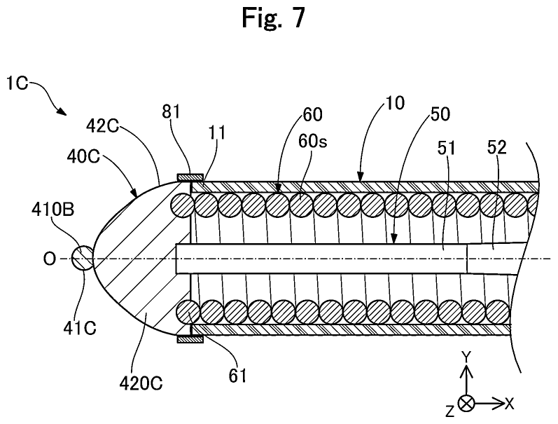

FIG. 7 is an enlarged sectional view illustrating a distal end side of a plasma guide wire 1C according to the fourth embodiment. The plasma guide wire 1C according to the fourth embodiment includes a distal tip 40C instead of the distal tip 40 in the configuration according to the first embodiment. The distal tip 40C has a first member 410C instead of the first member 410, and a second member 420C instead of the second member 420.

The first member 410C is disposed on the frontmost end side of the plasma guide wire 1C, and the second member 420C is disposed on the proximal end side relative to the first member 410C. The first member 410C has a spherical shape that a part on the proximal end side is cut out, and the second member 420C has a hemispherical shape. The first member 410C is fixed to the distal end of the second member 420C (in other words, the apex of the hemisphere constituting the second member 420C). The material for the first member 410C, the material for the second member 420C, and the method for fabricating the distal tip 40C (means for brazing, welding, soldering, plating, melting, etc.) are the same as those in the first embodiment. Similarly to the first embodiment, a distal end side region 41C is located on the distal end side relative to a proximal end side region 42C. Both the distal end side region 41C and the proximal end side region 42C have a smooth surface shape without any partially-sharpened corner portion.

As described above, the configuration of the distal tip 40C can be variously modified, and the distal end side region 41C may be arranged in any form as long as the distal end side region 41C is located on the distal end side relative to the proximal end side region 42C and the distal end side region 41C has an electric resistance value lower than of the proximal end side region 42C. For example, as illustrated in FIG. 7, the distal end side region 41C may be configured as an outer surface of a protruding portion where the distal tip 40C partially protrudes. The plasma guide wire 1C according to the third embodiment described above can also exhibit the same effects as those of the first embodiment described above.

### Fifth Embodiment

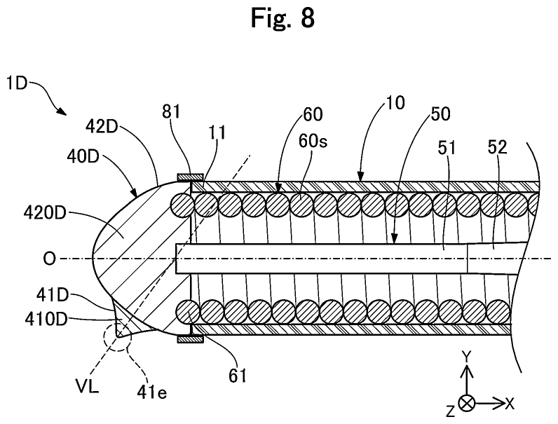

FIG. 8 is an enlarged sectional view illustrating a distal end side of a plasma guide wire 1D according to the fifth embodiment. The plasma guide wire 1D according to the fifth embodiment includes a distal tip 40D instead of the distal tip 40 in the configuration according to the first embodiment. The distal tip 40D has a first member 410D instead of the first member 410, and a second member 420D instead of the second member 420.

The first member 410D is disposed on the frontmost end side of the plasma guide wire 1D and the second member 420D is disposed on the proximal end side relative to the first member 410D. Since the proximal end of the first member 410D is located on the distal end side relative to the proximal end of the second member 420D, the second member 420D is defined as disposed on the proximal end side relative to the first member 410D in the fifth embodiment. The first member 410D has a conical shape, and the second member 420D has a hemispherical shape. The first member 410D is fixed to an intermediate portion between the apex and the edge of the second member 420D. The material for the first member 410D, the material for the second member 420D, and the method for fabricating the distal tip 40D (means for brazing, welding, soldering, plating, melting, etc.) are the same as those in the first embodiment. Similarly to the first embodiment, a distal end side region 41D is located on the distal end side relative to a proximal end side region 42D. Since the most proximal end portion of the distal end side region 41D is located on the distal end side relative to the most proximal end portion of the proximal end side region 42D, the distal end side region 41D is defined as located on the distal end side relative to the proximal end side region 42D in the fifth embodiment.

As illustrated in FIG. 8, a part of the outer surface of the distal end side region 41D (portion surrounded by the circular dashed line frame) has a corner portion 41e sharper than the other portion of the outer surface (portion outside the circular dashed line frame). As illustrated in FIG. 8, in the longitudinal section including the center axis O of the core shaft 50 and the corner portion 41e, the distal end of the corner portion 41e is oriented in a direction intersecting the center axis O of the core shaft 50. In FIG. 8, a virtual line VL extending in a direction in which the distal end of the corner portion 41e is oriented is represented by a dashed line. As illustrated, the virtual line VL (dashed line) intersects the center axis O (dashed-dotted line). The proximal end side region 42D has a smooth surface shape without any partially-sharpened corner portion.

As described above, the configuration of the distal tip 40D can be variously modified, and the distal end side region 41D may be arranged in any form as long as the distal end side region 41D is located on the distal end side relative to the proximal end side region 42D and the distal end side region 41D has an electric resistance value lower than of the proximal end side region 42D. For example, as illustrated in FIG. 8, the distal end side region 41D may be configured as an outer surface of a protruding portion where the distal tip 40D partially protrudes. The distal tip 40D may have the distal end side region 41D provided with the corner portion 41e. The plasma guide wire 1D according to the fifth embodiment described above can also exhibit the same effects as those of the first and second embodiments described above.

In the plasma guide wire 1D according to the fifth embodiment, the distal end of the corner portion 41e is oriented in a direction intersecting the center axis O of the core shaft 50 in the longitudinal section including the center axis O of the core shaft 50 and the corner portion 41e (FIG. 8). Thereby, a living body tissue in the vicinity of a blood vessel wall (in other words, a living body tissue oriented so as to intersect the traveling direction of the plasma guide wire 1D) can be ablated without pre-shaping the distal end portion of the plasma guide wire 1D.

### Sixth Embodiment

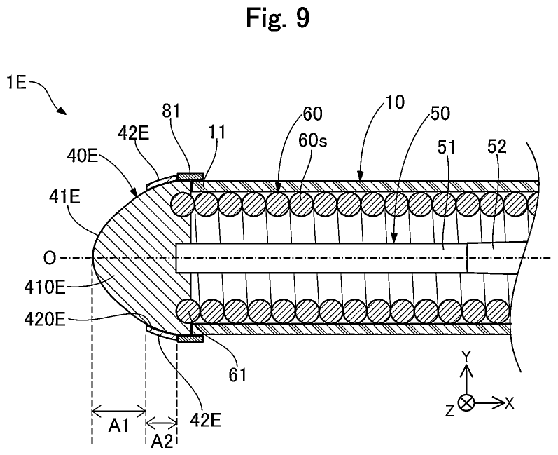

FIG. 9 is an enlarged sectional view illustrating a distal end side of a plasma guide wire 1E according to the sixth embodiment. The plasma guide wire 1E according to the sixth embodiment includes a distal tip 40E instead of the distal tip 40 in the configuration according to the first embodiment. The distal tip 40E has a first member 410E instead of the first member 410, and a second member 420E instead of the second member 420.

The first member 410E is a hemispherical member disposed on the frontmost end side of the plasma guide wire 1E. The second member 420E is an annular member. The second member 420E is fixed to the first member 410E while the second member 420E covers the outer surface of the first member 410E on the proximal end side. The material for the first member 410E, the material for the second member 420E, and the method for fabricating the distal tip 40E (means for brazing, welding, soldering, plating, melting, etc.) are the same as those in the first embodiment. Herein, as illustrated in FIG. 9, the outer surface of the first member 410E not covered with the second member 420E (in other words, the outer surface of the first member 410E exposed to the outside) on the outer surface of the distal tip 40E is also referred to as a distal end side region 41E, and the outer surface of the second member 420E is also referred to as a proximal end side region 42E. In FIG. 9, a portion corresponding to the distal end side region 41E is marked with symbol A1, and a portion corresponding to the proximal end side region 42E is marked with symbol A2. As illustrated in FIG. 9, the distal end side region 41E is located on the distal end side relative to the proximal end side region 42E. Both the distal end side region 41E and the proximal end side region 42E have a smooth surface shape without any partially-sharpened corner portion.

As described above, the configuration of the distal tip 40E can be variously modified, and the distal end side region 41E and the proximal end side region 42E may be arranged in any form as long as the distal end side region 41E is located on the distal end side relative to the proximal end side region 42E and the distal end side region 41E has an electric resistance value lower than of the proximal end side region 42E. In the example of FIG. 9, the first member 410E having the distal end side region 41E is configured in a form of the main body portion constituting the main body of the distal tip 40E, and the second member 420E having the proximal end side region 42E is configured in a form of the covering portion that covers the main body portion, but the contrary is possible. That means, the second member 420E having the proximal end side region 42E may be configured in a form of the main body, and the first member 410E having the distal end side region 41E may be configured in a form of the covering portion. The plasma guide wire 1E according to the sixth embodiment described above can also exhibit the same effects as those of the first embodiment described above.

### Seventh Embodiment

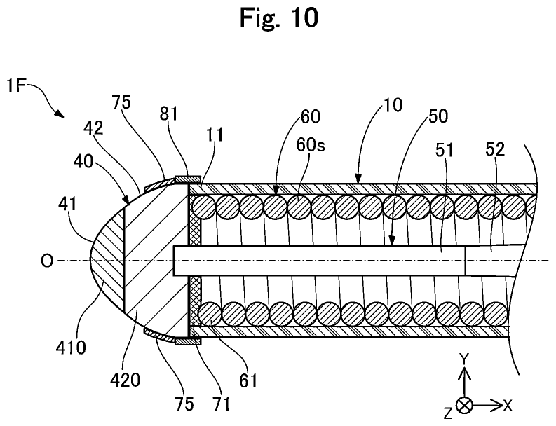

FIG. 10 is an enlarged sectional view illustrating a distal end side of a plasma guide wire 1F according to the seventh embodiment. The plasma guide wire 1F according to the seventh embodiment further includes a third fixation portion 71 and a covering member 75 in the configuration according to the first embodiment.

The third fixation portion 71 fixes the distal end portion 11 of the first tube 10, the distal end portion of the core shaft 50, and the distal end portion 61 of the coil body 60. The third fixation portion 71 is joined to the proximal end portion of the distal tip 40. The covering member 75 is an insulating annular member. The covering member 75 is joined to the second member 420 while covering the outer surface of the second member 420 on the proximal end side. Any bonding agent such as an epoxy adhesive may be used for joining the third fixation portion 71 and the covering member 75. Similarly to the first tube 10 and the second tube 20, the covering member 75 can be made of any insulating resin material. Instead of providing the covering member 75, a configuration in which the distal end portion 11 of the first tube 10 covers the outer surface of the second member 420 on the proximal end side may be adopted.

As described above, the configuration of the plasma guide wire 1F may be variously modified, and another member not described in the first embodiment may be adopted, and a part of the member described in the first embodiment may be omitted. The plasma guide wire 1F according to the seventh embodiment described above can also exhibit the same effects as those of the first embodiment described above. In the plasma guide wire 1F according to the seventh embodiment, since the proximal end side of the second member 420 is covered with the insulating covering member 75, the electric field intensity distribution generated around the distal tip 40 in association with streamer corona discharge can be further prevented from extending to the distal end portion 11 of the first tube 10. As a result, it is possible to provide the plasma guide wire 1F that allows ablation to be further localized, and further suppress a damage to the first tube 10.

### Eighth Embodiment

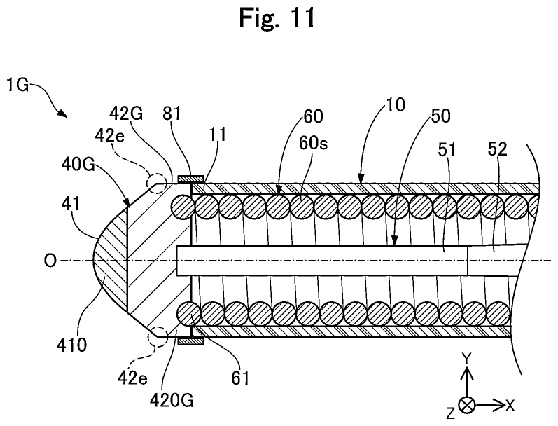

FIG. 11 is an enlarged sectional view illustrating a distal end side of a plasma guide wire 1G according to the eighth embodiment. The plasma guide wire 1G according to the eighth embodiment includes a distal tip 40G instead of the distal tip 40 in the configuration according to the first embodiment. The distal tip 40G has a second member 420G instead of the second member 420.

The second member 420G is disposed on the proximal end side relative to the first member 410. The second member 420G has a columnar shape combined with a truncated conical shape. The material for the second member 420G and the method for fabricating the distal tip 40G having the second member 420G (means for brazing, welding, soldering, plating, melting, etc.) are the same as those in the first embodiment. Similarly to the first embodiment, the distal end side region 41 is located on the distal end side relative to a proximal end side region 42G. As illustrated in FIG. 11, a part of the outer surface of the proximal end side region 42G (portion surrounded by the circular dashed line frame) is provided with a corner portion 42e sharper than the other portion of the outer surface (portion outside the circular dashed line frame). Since the corner portion 42e is formed at the boundary between the columnar portion and the truncated conical portion of the second member 420G, the corner portion 42e according to the eighth embodiment is formed over the entire circumferential direction of the plasma guide wire 1G. The distal end side region 41 has a smooth surface shape without any partially-sharpened corner portion.

As described above, the configuration of the distal tip 40G can be variously modified, and the proximal end side region 42G may have the corner portion 42e. The plasma guide wire 1G according to the eighth embodiment described above can also exhibit the same effects as those of the first embodiment described above.

### Ninth Embodiment

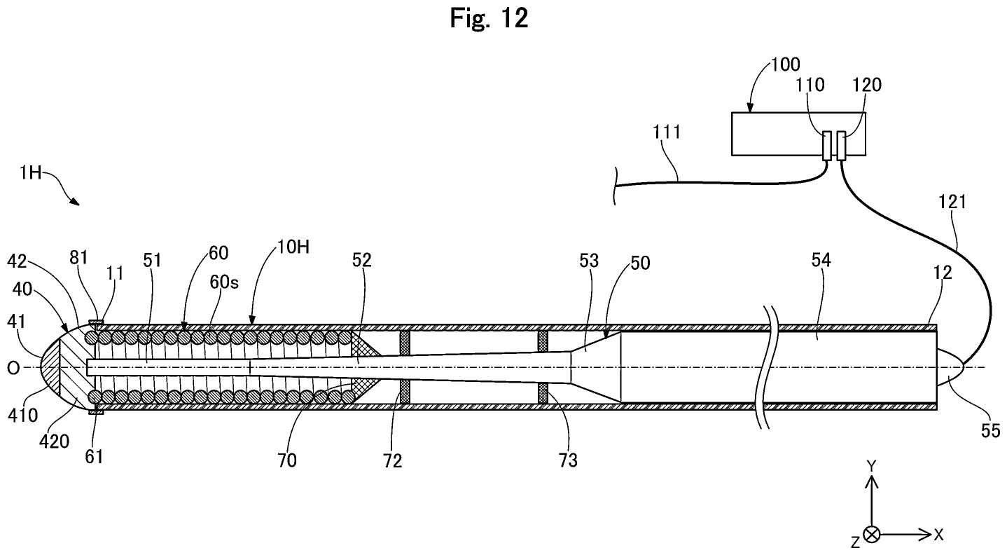

FIG. 12 is an explanatory view illustrating a sectional configuration of a plasma guide wire 1H according to the ninth embodiment. The plasma guide wire 1H according to the ninth embodiment includes a first tube 10H instead of the first tube 10, the second tube 20, and the third tube 30 in the configuration according to the first embodiment. The first tube 10H is a hollow cylindrical-shaped columnar member that covers the whole guide wire main body, i.e., the outer periphery of the coil body 60 and the outer periphery of the core shaft 50 excluding the proximal end portion 55.

As described above, the configuration of the plasma guide wire 1H can be variously modified, and the guide wire main body may be covered with the single first tube 10H. The guide wire main body may be covered with two or four or more tubes in combination with each other in the longitudinal direction of the plasma guide wire 1H. The plasma guide wire 1H according to the ninth embodiment described above can also exhibit the same effects as those of the first embodiment described above. The configuration of the plasma guide wire 1H according to the ninth embodiment can be simplified to reduce the manufacturing cost.

### Modification Examples of Embodiments

The disclosed embodiments are not limited to the above-described embodiments and may be implemented in various modes without departing from the gist thereof, and for example, the following modifications are also possible.

### Modification Example 1

In the first to ninth embodiments, examples of the configurations of the plasma guide wires 1 and 1A to 1H have been described. However, the configurations of the plasma guide wires 1 and 1A to 1H can be variously modified. For example, in the distal tips 40, 40A to 40E, and 40G, the surface area of the distal end side region 41 may be the same as or larger than the surface area of the proximal end side region 42. For example, in the distal tips 40, 40A to 40E, and 40G, corner portions may be provided on both the distal end side region 41 and the proximal end side region 42. For example, the distal tips 40, 40A to 40E, and 40G may further have, between the distal end side region 41 and the proximal end side region 42, an intermediate region having an electric resistance value different from those of the distal end side region 41 and the proximal end side region 42.

For example, the core shaft 50 constituting the guide wire main body is not limited to the above-described shape but may have any shape. For example, at least a part of the small diameter portion 51, the first tapered portion 52, the second tapered portion 53, the large diameter portion 54, and the proximal end portion 55 described as examples in the above embodiments may be omitted. For example, the guide wire main body may include additional configurations not described above. For example, an inner coil body may be provided inside the coil body 60.

### Modification Example 2

The configurations of the plasma guide wires 1 and 1A to 1H according to the first to ninth embodiments and the configurations of the plasma guide wires 1 and 1A to 1H according to Modification Example 1 may be combined with each other as appropriate. For example, the plasma guide wire 1 according to the second to sixth, eighth, and ninth embodiments may include the third fixation portion 71 and the covering member 75 described in the seventh embodiment. For example, the plasma guide wire 1 according to the second to eighth embodiments may include the first tube 10H described in the ninth embodiment.

Although the aspects of the disclosed embodiments have been described above on the basis of the embodiments and modification examples, the embodiments of the aspects described above are intended to facilitate understanding of the aspects, and are not intended to limit the aspects. The aspects may be modified and improved without departing from the gist and the scope of claims and includes equivalents thereof. If the technical features are not described as essential in the present specification, the technical features may be appropriately deleted.

The disclosed embodiments can be embodied as the aspects described below.

### Application Example 1

A plasma guide wire including:
a conductive core shaft;
a conductive coil body that surrounds a part of the core shaft on a distal end side; and
a distal tip made of a conductive metal material, which fixes a distal end of the core shaft and a distal end of the coil body and receives a high frequency wave from a high-frequency generator electrically connected to the core shaft, in which
an outer surface of the distal tip includes a proximal end side region located on a side of the coil body and a distal end side region located on a distal end side relative to the proximal end side region, and
the distal end side region has a lower electric resistance value than of the proximal end side region.

### Application Example 2

The plasma guide wire according to Application Example 1, in which
a part of the outer surface in the distal end side region has a corner portion sharper than the other portion.

### Application Example 3

The plasma guide wire according to Application Example 1 or 2, in which
the proximal end side region does not have the corner portion.

### DESCRIPTION OF REFERENCE NUMERALS

1 and 1A to 1H: Plasma guide wire
1x: Plasma guide wire (comparative example)
10 and 10H: First tube
20: Second tube
30: Third tube
40, 40A to 40E, and 40G: Distal tip
40x: Distal tip (comparative example)
41 and 41A to 41E: Distal end side region
41e: Corner portion
42, 42B to 42E, and 42G: Proximal end side region
42e: Corner portion
50: Core shaft
51: Small diameter portion
52: First tapered portion
53: Second tapered portion
54: Large diameter portion
55: Proximal end portion
60: Coil body
60s: Wire
70: Coil fixation portion
71: Third fixation portion
72: First fixation portion
73: Second fixation portion
75: Covering member
81: Distal end marker
100: RF Generator
110: First terminal
111: First cable
120: Second terminal
121: Second cable
410 and 410A to 410E: First member
420, 420B to 420E, and 420G: Second member

What is claimed is:

1. A plasma guide wire comprising:
a conductive core shaft configured to be connected to a high-frequency generator;
a conductive coil body that surrounds a part of the core shaft on a distal end side of the core shaft; and
a distal tip that includes a conductive material and fixes a distal end of the core shaft and a distal end of the coil body, the distal tip being configured to receive a high frequency wave from the high-frequency generator via the core shaft, wherein
an outer surface of the distal tip includes (i) a proximal end side region located on a side of the coil body and (ii) a distal end side region located distally relative to the proximal end side region, and
the distal end side region has an electric resistance value that is lower than an electric resistance value of the proximal end side region.

2. The plasma guide wire according to claim 1, wherein
a portion of the outer surface of the distal tip in the distal end side region has a corner portion that is sharper than a remaining portion of the outer surface of the distal tip in the distal end side region.

3. The plasma guide wire according to claim 2, wherein
the proximal end side region does not have any corner portion.

4. The plasma guide wire according to claim 1, wherein
the distal tip includes a distal end marker indicating a position of the distal tip, the distal end marker being insulative.

5. The plasma guide wire according to claim 1, wherein
the distal tip fixes the distal end of the core shaft and the distal end of the coil body in only the proximal end side region.

6. The plasma guide wire according to claim 5, wherein
the distal end of the core shaft and the distal end of the coil body are embedded in the distal tip in the proximal end side region.

7. The plasma guide wire according to claim 1, wherein
a surface area of the distal tip in the distal end side region is smaller than a surface area of the distal tip in the proximal end side region.

8. The plasma guide wire according to claim 1, wherein
an interface between the distal end side region and the proximal end side region of the distal tip extends in a direction perpendicular to a central longitudinal axis of the plasma guide wire.

9. The plasma guide wire according to claim 1, wherein
an interface between the distal end side region and the proximal end side region of the distal tip extends in a direction that is not perpendicular to a central longitudinal axis of the plasma guide wire.
